(19) United States
(12) Patent Application Publication
(10) Pub. No.: US 2015/0369788 A1
(43) Pub. Date: Dec. 24, 2015

(54) Sensor Device, Filter Assembly, and Method for Manufacturing Same

(71) Applicant: MANN+HUMMEL GMBH, Ludwigsburg (DE)

(72) Inventor: Thomas Andres, Bobenheim/Roxheim (DE)

(21) Appl. No.: 14/742,161

(22) Filed: Jun. 17, 2015

(30) Foreign Application Priority Data
Jun. 20, 2014 (DE) 10 2014 008 830.8

(51) Int. Cl. G01N 33/28; B01D 35/00; B29C 45/14; B01D 35/31

(52) U.S. Cl. CPC G01N 33/2847; B01D 35/31; B01D 35/005; B29C 45/14819; B29K 2075/00

(57) ABSTRACT

A sensor device for a filter assembly is provided with at least one sensor element that detects an operating parameter of the filter assembly. A fire protection material at least partially encloses the sensor element. The sensor element is a water sensor. The fire protection material comprises an intumescent material.

### BACKGROUND OF THE INVENTION

The present invention concerns a sensor device for a filter assembly such as for an operating medium filter in a vehicle. Moreover, the invention concerns a method for manufacturing such a sensor device.

In certain filter applications, the automatic detection of the substances separated from each other is desired. For example, as is known from WO2012/025405A1, in case of fuel filters, in particular for diesel injection engines, water components are separated from or filtered out of the diesel fuel. The separated water is in general collected and its filling level is detected by means of a moisture sensor or water sensor. A reliable function of the filter assembly and/or of the corresponding sensor even under special operating conditions is desirable in this context. The sensors or sensor devices are in general provided separate from the actual filter element. For example, a sensor with a measuring head that extends into the interior of the filter element can be connected fluid-tightly with the filter element so that operating parameters of the filter element can be detected during operation. In this context, the actual sensor device is arranged outside of the filter element, for example, screwed on, connected by a flange, or pushed on.

### SUMMARY OF THE INVENTION

In view of this background, the present invention has the object to provide an improved sensor device.

Accordingly, a sensor device for a filter assembly is proposed which comprises at least one sensor element for detecting an operating parameter for the filter assembly and comprises a fire protection material which at least partially surrounds the sensor element.

For example, the fire protection material protects the sensor element from high temperatures which may impair the function of the sensor element or a coupling action of the sensor element with the filter assembly. In embodiments, the fire protection material surrounds the sensor element completely or to a large part. The fire protection material can also be formed such that it comprises a receiving area for the sensor element.

The fire protection material shields in this context the sensor element from flames or heat acting as a result of fire from the exterior on the filter assembly in such a way that for a predetermined amount of time and for predetermined flame temperatures the function of the sensor element and/or a sealing action of the sensor element with the inserted filter remains unaffected. An operating parameter is, for example, the presence of water in or on the filter assembly. Conceivable is however also the configuration of the sensor element for detecting fluids, such as gases or liquids, temperatures, pressures, electrical conductance or the like. The sensor element is in particular a water sensor.

In embodiments, the sensor device is in particular suitable for a filter assembly that is configured for filtering operating media in vehicles, such as motor vehicles, water craft, rail vehicles, stationary electrical generators or aircraft. The sensor device is in particular mounted in or on a filter such as a fuel filter, a diesel fuel filter for diesel injection engines. One can speak also of pre-line filters or prefilters which are provided with an exchangeable filter for particle and water separation of fuels. Filters that can be exchanged as a whole, are mountable on a filter head and comprise a closed housing, usually made of metal, with a filter body arranged therein are referred to as exchangeable filter. These filters are also known as spin-on filters because they are usually mountable on the filter head by means of a thread.

In embodiments, the fire protection material comprises an intumescent material. Intumescent materials expand upon thermal loading or foam and form a thick heat-insulating and flame-insulating protective layer. Intumescent materials are also known as fire protection foams. As fire protection material, in particular a polyurethane foam is conceivable which has fire protection additives added to it. The fire protection material can be present as a shaped part in a suitable geometry in the sensor device. It is also possible to embed by foaming the sensor element with appropriate foam material. Preferably, the intumescent material has a density of less than 1,000 kg/m³, in particular less than 400 kg/m³.

In further embodiments, the fire protection material comprises a flame-resistant fiber material. For example, glass filament fabrics are known which can be used as heat shields. The fire protection material can comprise, for example, silicate glass fibers which have a filament diameter of >6 μm. In particular mineral or glass fiber materials are temperature-resistant, in general up to 1,000° C., and have good insulating properties. In particular needle mats as fire protection materials are non-combustible and have an excellent vibration resistance. A fire protection material comprising fiber material can be manufactured as a shaped part about the respective sensor element. It is possible to attach the fire protection material with fastening means such as clamps, housing parts, nets or other means that enable a spatial fixation on or about the sensor element.

The fire protection material can moreover comprise a ceramic material. Ceramics can be used, for example, as a potting compound and can surround the sensor element at least partially. The ceramic material comprises in particular an aluminum and/or calcium compound. As ceramic materials, the following are suitable: corundum with calcium aluminate bond, aluminum oxide, calcium oxide, magnesium oxide, iron oxide as well as mixtures of the aforementioned materials.

In one embodiment, the fire protection material is a stainless steel material forming the housing of the sensor element and enclosing it completely. Heat that is acting on the housing can be dissipated partially via the sensor element to the water contained in the interior of the filter and the sensor; this delays the heating process in case of fire. In this context, the housing is preferably provided with an annular disk-shaped ring projection in an area adjoining the filter and projects distinctly in radial direction (i.e., at least 5 mm and preferably at least 10 mm) past the connecting area of the sensor element with connecting thread and seal. In this way, flames can be deflected so that the risk of a direct contact of the connecting area with the flames is significantly reduced.

In embodiments, at least one cover plate is attached to the fire protection material. For example, the fire protection material can be arranged between two cover plates. A carbon material can be used as a cover plate material. In particular carbon fiber or polyacrylonitrile (PAN) fiber materials are suitable as cover plate material. The thickness of the cover plate is, for example, between 1 and 3 mm. Expandable graphite foams, for example, already at 145° C. and expands. Graphite has in particular the advantage that it is aging-resistant, water resistant, and UV resistant.

In further embodiments, the fire protection material and/or the cover plate material comprises an expandable graphite.

In embodiments, a cylinder-shaped fire protection material is provided between two cover plates that form the cover surfaces.

In embodiments, the sensor element is surrounded by an enclosing fire protection housing. The fire protection housing is configured for receiving the fire protection material about the sensor element. For example, the sensor element, on the one hand, can adjoin the filter assembly or a filter and, on the other hand, can be surrounded by a fire protection housing which provides protection in case of flame action from the exterior. The fire protection housing can be manufactured of a metal material. The fire protection housing is, for example, provided with a handling section in order to separate a unit comprised of the sensor element, the fire protection material, and the housing from the filter of the filter assembly.

In embodiments, the housing has openings. For example, material that foams in case of fire with volume enlargement can exit through the openings and therefore better protect the sensor element. It is also possible that within the fire protection housing the sensor element and the fire protection material are connected to each other in such a way that a destruction-free detachment of the parts from each other is impossible.

In embodiments of the sensor device, a cavity is provided between the fire protection material and the sensor element. This cavity or a gap between the outer surface of the sensor element and the fire protection material leads to an improved insulation relative to the exterior.

The sensor device may comprise in embodiments a sensor element in which at least one connecting line for transmitting sensor signals is provided. The connecting line is at least partially passing through the fire protection material to the exterior. The connecting line is, for example, a metal wire which is provided with plastic insulation. The connecting line is in particular a cable in a flame-protected configuration. For example, the cable fulfills DIN 5510 with fire protection level 1, 2, 3 or 4.

In embodiments, the sensor device is furnished with a fire protection material such that the sensor element withstands flame exposure of the sensor device for a predetermined amount of time at a predetermined temperature. Conceivable is, for example, that the predetermined amount of time is at least 20, preferably at least 30 minutes, and even more preferred 40 minutes. The flame temperature is, for example, at least 700° C., preferably 800° C., and further preferred 900° C. It is in particular conceivable that the sensor device is designed to withstand a fire resistance test according to ISO 7840, ISO 19 921 or according to the regulations of the shipping industry classification associations.

The sensor device for the filter assembly is in particular furnished such that at a connection between the sensor element and the filter assembly, such as, for example, a filter or spin-on filter, no leakage occurs. A coupling between the filter assembly and the sensor element is preferably fluid-tight in such a way that no leakage will occur at pressure differences within a single-digit bar range.

Moreover, a filter assembly with a filter, especially a spin-on filter, for filtering a fluid comprising a sensor device as described above is proposed. The sensor element is mounted fluid-tightly on the filter.

Preferably, a sealing device, for example, in the form of a sealing ring, is provided between the sensor element and the filter.

The filter assembly is in particular designed for use in water craft and rail vehicles and for filtering fuel for an internal combustion engine. The sensor element serves in this context for detecting water in a receiving space of the filter or the filter assembly.

The filter assembly may comprise in embodiments a securing device for the filter, the fire protection housing and/or an expanding fire protection medium. As a securing device, in particular clamps, webs, beads, grooves, openings or undercuts are suitable. For example, a fire protection housing for the sensor element can be attached by means of a clamp and one or several holding tabs on the filter and/or on the filter head.

Finally, a method for manufacturing a sensor device as described above is proposed. In this context, a sensor element is embedded at least partially with an (initially) fluidic fire protection material. The fire protection material is intumescent in a cured solidified state.

The method is suitable in particular for simple manufacture of sensor devices that fulfill requirements in regard to fire resistant systems, in particular in water craft and rail vehicles. In embodiments, for example, the sensor element is potted or embedded by injection-molding with a fire protection foam. It is conceivable to provide a cup-shaped fire protection housing which is filled with uncured fire protection material, and the sensor element is then inserted. Moreover, it is conceivable to provide a casting mold, to position the sensor in the mold, and to subsequently meter fire protection foam material into the casting mold that, upon foaming, encloses and also bonds to the sensor element and particularly preferred also to the area, adjoining the sensor element, of a connecting cable and of a water drainage pipe for water separated by the filter. Preferably, the fire protection foam after curing is connected in a torque-proof connection (in regard to torque usually occurring during manipulation by hand) with the sensor element such that the sensor element can by screwed into a thread provided for this purpose on the (spin-on) filter by a turning movement acting on the fire protection foam. For this purpose, preferably form fit elements can be provided externally on the sensor element. For example, the sensor element can have on its exterior side (in radial direction outwardly in relation to the center axis) a polygonal shape or can be provided with ribs, knurling, or other structures, suitable to transmit torque, that are form-fittingly surrounded by the fire protection foam. Preferably, the water drainage pipe projects spatially in downward direction from the fire protection foam. This facilitates handling as well as manufacture.

In embodiments of the filter assembly, the filter has an inlet and an outlet and serves for filtering operating media for internal combustion engines. The filter can have a cylindrical shape and, when properly installed, a water drainage opening as well as a connector for a sensor element are provided at its bottom side.

A housing of the spin-on filter or of the filter is, for example, manufactured of a fire-resistant material with a melting point greater than 900° C., such as a metal material. Moreover, the filter assembly can be designed, for example, like a pre-filter system with a filter head that optionally comprises a pump, a heating element, and hose connectors for inlet and outlet. The filter assembly fulfills in particular a pre-filter function with water separation. The filter assembly is operated at an operating pressure of −1 to 7 bar or 3 to 5 bar. Accordingly, fluid-tight transitions between filter, filter head, and the connected sensor element are designed for overpressure or negative pressure up to 8 bar.

A corresponding filter is provided with a filter body of a filter medium. The filter medium can be a flat material that is embodied to be folded or corrugated. As folds, for example, zigzag folds or W folds are known. The filter medium can be embossed and subsequently can be folded sharply at embossed edges with formation of fold edges. As starting material, a flat filter material sheet can be provided which is then appropriately reshaped. An endless fold bellows can be formed.

The filter medium is, for example, a filter fabric, a laid filter, or a filter nonwoven. In particular, the filter medium can be produced by a spun-bonded fabric or meltblown method. Moreover, the filter medium can be felted or needled. The filter medium can comprise natural fibers, such as cotton, or synthetic fibers, for example, of polyester, polyvinyl sulfite or polytetrafluoroethylene. The fibers can be oriented during manufacture in the machine direction, at a slant thereto, and/or transverse thereto. The filter medium can be of a single-layer or a multi-layer configuration. The medium to be filtered is, for example, air. A gaseous medium or air is to be understood herein also as mixtures of gas or air with solid material and/or gas or air with liquid. Likewise, operating liquids such as fuels, lubricants, urea solutions and the like are conceivable as media to be filtered.

The filter can be used in passenger cars, trucks, construction machinery, water craft, rail vehicles, aircraft, as well as generally in air-conditioning technology, in particular in heating air-conditioning devices, in household appliances, in fuel cells or in building technology. Said vehicles can be electrically operated and/or by means of fuel (in particular gasoline, diesel or natural gas). In regard to building technology, in particular stationary devices for treatment of air, for example, for stationary electric power generation, are conceivable.

Further possible implementations of the invention comprise also combinations, not explicitly mentioned, of features or method steps described above or in the following with regard to the embodiments. In this context, a person of skill in the art will also add individual aspects as improvements or supplements to the respective basic form of the sensor device or of the filter.

Further embodiments of the invention are subject matter of the dependent claims as well as of the embodiments described in the following. In the following, the invention will be explained in more detail with the aid of embodiments with reference to the attached figures.

### BRIEF DESCRIPTION OF THE DRAWING

FIG. 1 shows a schematic section view of a first embodiment of a filter assembly with a sensor device.

FIG. 2 shows a schematic detail section view of the sensor device.

FIG. 3 is a perspective view of the sensor device.

FIG. 4 is a perspective view of a second embodiment of the filter assembly with sensor device.

FIG. 5 is a side view of the filter assembly of FIG. 4.

FIG. 6 is a detail section view of the sensor device of the second embodiment.

FIG. 7 is a perspective view of the sensor device of the second embodiment.

FIG. 8 is a schematic section view of a third embodiment of a sensor device.

FIG. 9 is a perspective view of the sensor device of the third embodiment.

FIG. 10 is a schematic partial section illustration of a fourth embodiment of a sensor device.

### DESCRIPTION OF PREFERRED EMBODIMENTS

In the following, embodiments of filter assemblies and sensor devices will be described with the aid of prefilters for diesel fuel. In this context, in particular a water sensor is provided as a sensor element. Other filter assemblies, similar to the embodiments, with deviating operating media, filter media, or sensors can be used.

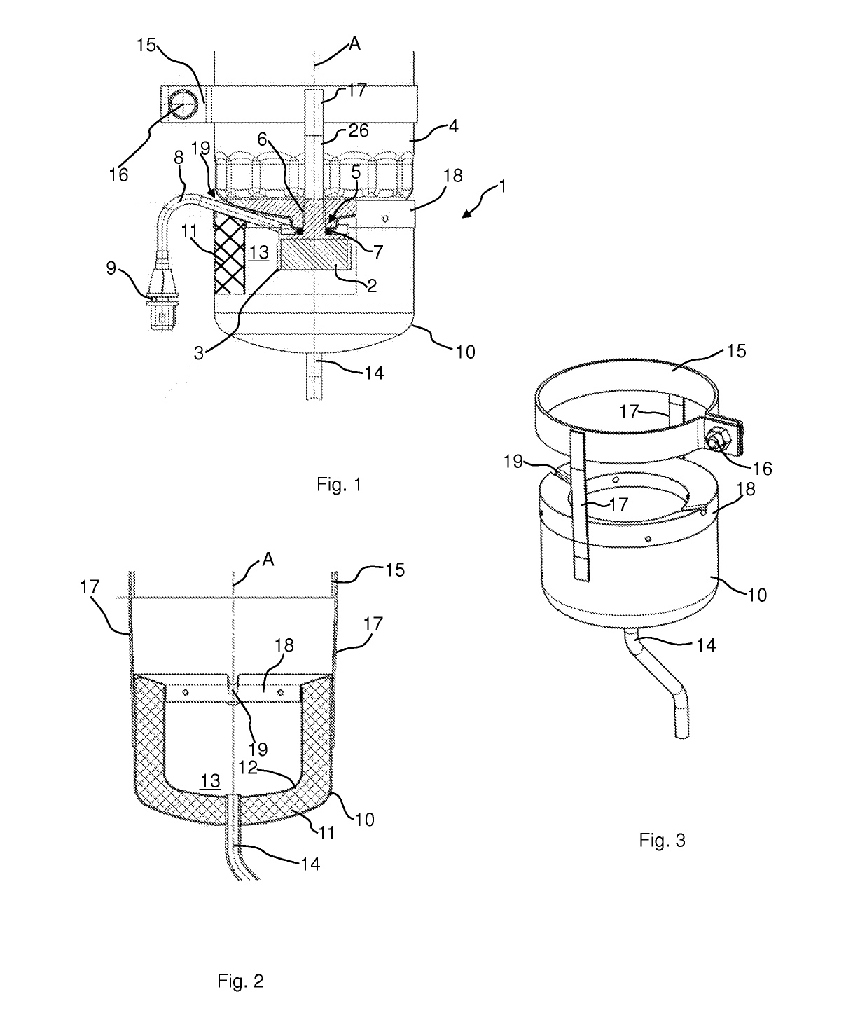

FIGS. 1-3 show schematic section view and perspective illustrations of a first embodiment of a sensor device 1. FIG. 1 shows in this context a section view of the sensor device 1 which is coupled to a fluid filter 4 embodied as a spin-on filter. FIG. 2 shows a section view of a device section that is acting as a fire protection housing on the sensor device, and FIG. 3 a perspective illustration thereof.

In the upper area of FIG. 1 a spin-on filter 4 is schematically illustrated. The spin-on filter 4 is cylindrical and has in the orientation of FIG. 1, which reflects the typical mounting situation, an opening 5 at the bottom for connecting a sensor element 2. The spin-on filter 4 comprises in this context, for example, a filter cup of a metal material. The spin-on filter 4 serves in this context for filtering fuel and separating water from the fuel.

In operation of the spin-on filter 4, in the lower area the separated water is collecting as a liquid. In order to be able to detect the filling level or the presence of water, a sensor device 2 is provided. The sensor device 2 is accommodated in a sensor housing 3 that is usually made of plastic material and projects with a measuring head 26 into the interior of the filter cup of the filter 4.

The sensor element 2 is fluid-tightly connected with the housing of the spin-on filter 4. For this purpose, a sealing ring 7 is provided that provides in particular an axial sealing action along the center axis A of the resulting filter assembly with the sensor device 1. Alternatively, a sealing action in radial direction or at a slant with radial and axial proportions can be realized also. For connecting the sensor element 2 to the spin-on filter 4, a threaded member 6 can be provided or other connecting means that are not illustrated here. In operation of the filter or spin-on filter 4, pressures of up to 8 bar result, i.e., the closure of the spin-on filter 4 at the opening 5 resists these pressures by means of the sensor element 2.

By means of a connecting cable 8, herein extending laterally outwardly, and a plug connector 9 connected thereto, the sensor element 2 supplies sensor signals to an evaluation unit. The latter can be, for example, a vehicle electronic unit or an evaluation circuit. It is also conceivable that the sensor device 1 supplies signals to a vehicle bus, for example, a CAN bus.

The sensor element 2 is surrounded outwardly by a fire protection housing 10 which is designed to be sleeve-shaped or cup-shaped. The housing is illustrated in FIG. 3 in a perspective view and comprises a lower fire protection housing part 10 that is designed to be cup-shaped or pot-shaped. An upper transition member 18 is provided with a passage 19 for the connecting cable 8 and contacts snugly the contour of the lower area of the spin-on filter 4. This can be seen in particular in FIG. 1.

The fire protection housing 10 is attached with a clamp 15 and one or two securing strips 17 on the spin-on filter 4. For this purpose, the clamp 15 designed as a hose clamp is attached by means of screwing means 16. At the bottom side on the fire protection housing cup 10, a drain or drainage pipe 14 is provided through which the liquid present in the interior of the fire protection housing 10 can drain. The drain 14 is designed to be angled and is bent away laterally. Accordingly, potential flames cannot penetrate, or hardly penetrate, through the angled pipe or the drain 14.

In FIG. 1 a view into the fire protection housing 10 is indicated by dashed lines. FIG. 2 shows a longitudinal section through the fire protection housing 10 in which the sensor element 2 is received. In this context, the fire protection housing 10 is provided with a fire protection material 11 which surrounds like a cup the sensor element 2. At the top side, the fire protection material 11 adjoins the bottom side of the spin-on filter 4. In the mounted state, one can see in FIG. 1 that a cavity 13 is existing between the sensor housing 3 and the inner side 12 of the fire protection housing 10. The hollow space 13 or the gap serves as a further insulation in a fire situation.

The fire protection material 11 which is illustrated in crosshatching comprises, for example, a mineral, a ceramic potting compound or glass fiber material which resists high temperatures and exposure to flames. This can also be referred to as flame protection enclosure of the sensor element 2.

Alternatively or additionally, the fire protection material 11 may comprise a fire protection foam which upon heat exposure swells or expands and thus will keep flames or heat entering from the exterior away from the sensor element or the seal 7. The sensor device 1 is in particular encased in such a way with the fire protection housing 10 that it will pass a flame protection test according to the afore described regulations. This means that no filter fluid will escape from the interior of the spin-on filter 4 even after a 30-minute flame exposure or a temperature increase to more than 850° C.

In the embodiments, in particular materials that have a melting point of less than approximately 930° C. are encased with thefire protection material. The elements of the sensor device 1 which serve for attachment of the fire protection material 11 are, for example, manufactured of a metal material. These are in particular the clamp 15, the securing strip 17, the fire protection housing cup 10 as well as the drainage pipe 14.

The proposed sensor device 1 enables in particular an operation of the appropriately furnished pre-filter assemblies for diesel engines in marine applications in water craft. The actual sensor 2 can be manufactured with a sensor housing 3 of a polyamide material.

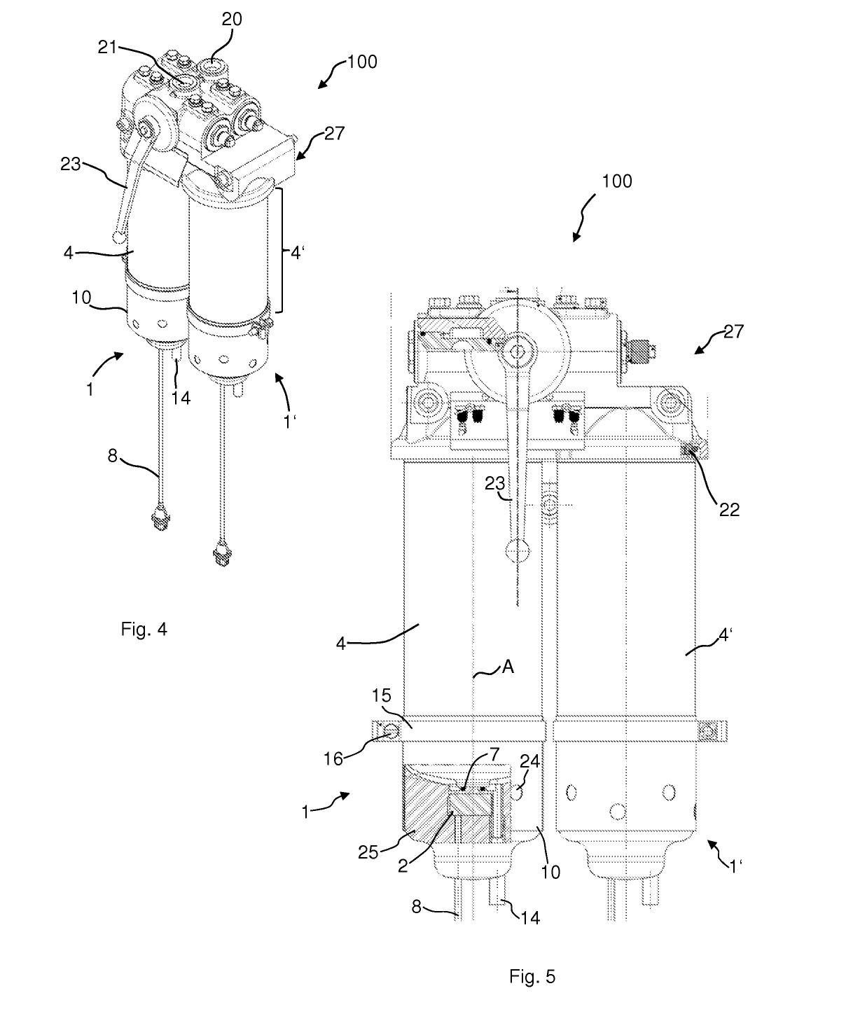

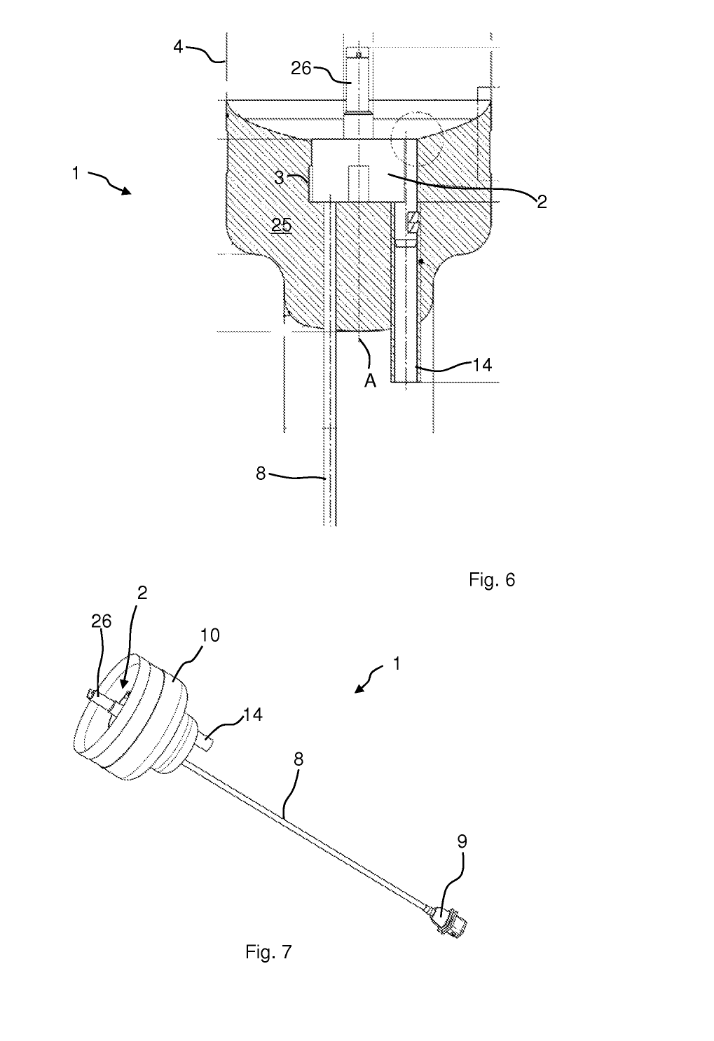

FIGS. 4 to 7 show schematic section views and perspective illustrations of an embodiment of a filter device and elements arranged therein. In this context, FIG. 4 shows a perspective view of the filter assembly, FIG. 5 a side view of the filter assembly, FIG. 6 a section view of the inserted sensor device, and FIG. 7 a perspective view of the embodiment of the sensor device. The elements of the sensor device which have been described in FIGS. 1-3 can be used identically or similarly herein as well.

FIG. 4 shows a perspective illustration of a prefilter assembly 100, in particular for use in diesel engines in or on water craft. The filter assembly 100 is furnished as a double filter with two spin-on filters 4, 4′. The filter assembly has in this context a filter head 27 that in particular comprises an inlet 20 for the fluid to be cleaned, such as diesel fuel, and an outlet for cleaned fuel 21. On the filter head 27 various fixtures and connecting pieces for the spin-on filters 4, 4′ are provided which will not be discussed in more detail in the following.

A lever 23 enables the activation and deactivation of the two spin-on filters 4, 4′ independent of each other. For example, in the lever position 23 to the left (compare FIG. 5) the left spin-on filter 4 can be decoupled from fuel and filter circulation and can be exchanged. By means of the two independently operable spin-on filters 4, 4′, during operation of the filter or the filter assembly 100 an exchange of an individual spin-on filter can be realized without interrupting operation. However, individual arrangements with or without shut-off valve are conceivable.

The spin-on filters are connected by means of sealing rings 22, as indicated in FIG. 5, on the filter head 27 in a fluid-tight manner. In the following, the left combination of spin-on filter 4 and sensor device 1 will be discussed essentially.

In FIG. 5, a sensor element 2 which is surrounded by a fire protection foam 25 (cross-hatched illustration), in particular is embedded in the fire protection foam 25, is shown that is coupled by a seal 7 to the spin-on filter 4. The fire protection foam 25 is embodied preferably as a shaped body of inherent stability. The fire protection foam 25 is surrounded radially outwardly by a fire protection housing 10 that is embodied as a tubular cylinder, for example, made of sheet steel, in particular of stainless sheet steel. The fire protection housing 10 is connected to the spin-on filter 4, 4′ and slightly spaced apart (approximately 0.3-2 mm) from the fire protection foam 25 so that the latter can be arranged rotatably within the fire protection housing 10. This rotating action is desirable for mounting the unit comprised of fire protection foam and sensor element and is required for regular drainage of the water separated within the spin-on filter 4, which is accomplished by loosening the sensor element. The fire protection housing 10 is attached by a clamp 15 and screws 16 to the spin-on filter 4. The fire protection material 25 is in this context a polyurethane foam which, for example, comprises halogen-free fire protection additives such as Clariant Exolit OP 560 or Lanxess Uniplex FRP and has intumescent properties. When exposed to flames from the exterior, the fire protection foam expands and deforms through the openings 24 provided within the fire protection housing 10. Due to the volume increase, the expanded fire protection foam protects the inwardly positioned sensor element 2 for a certain amount of time from destruction or combustion. Before a flame reaches the sensor element 2 and the seal 7, the expanded fire protection foam 25 will burn off first.

In FIG. 5, a broken-away window is illustrated in dash-dotted line at the lower left on the sensor device 1. The sensor element 2 comprises a connecting cable 8 that is guided, eccentric to the center axis A of the filter assembly 100 and of the sensor element 2, downwardly through the fire protection foam 25. Moreover, a water or liquid drainage passage 14 is also extending eccentrically relative to the center axis A.

In FIG. 6, a detail of the left sensor and filter device 1 is illustrated in section. The sensor element 2 is provided with a measuring head 26 which is extending into the interior of the spin-on filter 4. The connection between the interior of the spin-on filter 4 and the sensor element 2 is fluid-tight. Laterally relative to the sensor element 2, a water drainage passage 14 can be extended through the area of the fire protection material 25 to the exterior. The fire protection material 25 will get caught, for example, upon expansion, at the openings 24 (FIG. 5) of the fire protection housing 10. Moreover, further means, for example, rough surfaces, protrusions or projecting sheet metal areas below the opening 24 can be provided which impairs dropping or dripping of the fire protection foam that has exited.

FIG. 7 shows a perspective illustration of the sensor device 1 as it is used in the embodiment for the filter assembly according to FIGS. 4 and 5. The pot-shaped or cup-shaped fire protection housing 10 that is, for example, made from sheet metal is preferably filled with the fire protection foam 25. In FIG. 7, one can see the connecting cable 8 with plug connector 9 as well as the water drain 14 exiting from the area of the fire protection foam.

In proper use, the center axis or symmetry axis A of the filter assembly 100 and/or of the sensor device 1 points vertically downwardly, i.e., along the gravitational acceleration. Accordingly, water can exit downwardly through the drain 14. Due to the flame protection or fire protection material about the sensor device or the sensor element 3 usually made of plastic material, a reliable operation can still be ensured for a certain amount of time in case of a fire in the vicinity of the filter assembly 100.

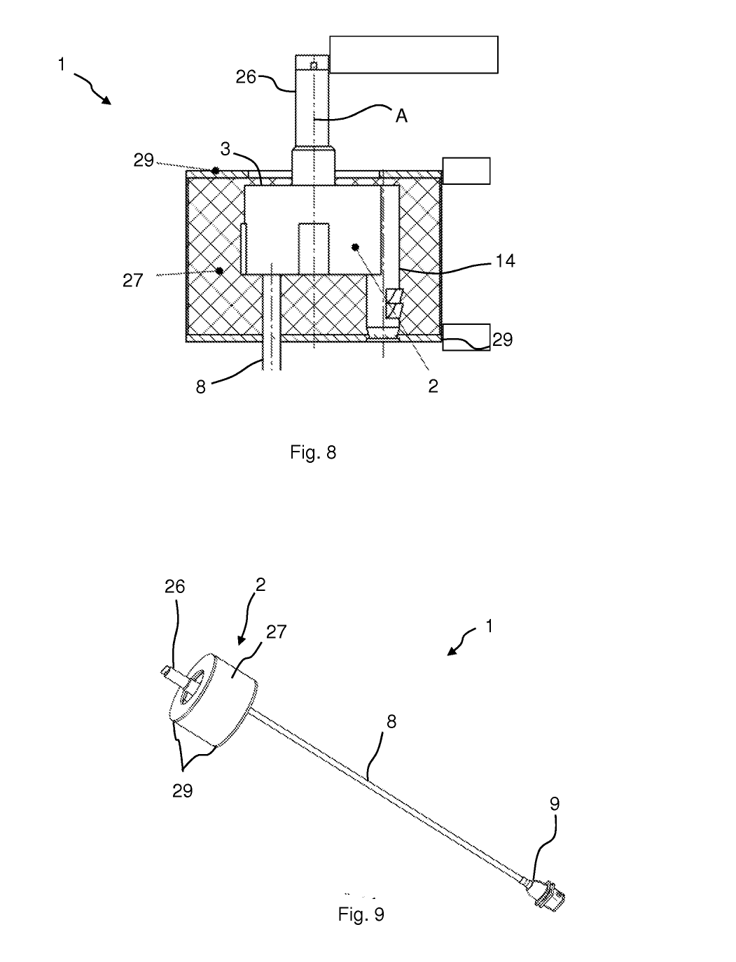

FIGS. 8 and 9 show a third embodiment of a sensor device 1. FIG. 8 shows a longitudinal section of the sensor device similar to FIG. 6, and FIG. 9 is a perspective illustration of the sensor device. Essentially the same elements provided with the same reference characters as in the first and second embodiment are present and no discussion in more detail will be provided in this context. The fire protection encasement of the sensor element 2 is realized by a ceramic potting compound 27 such as a fire-resistant potting compound on the basis of corundum with calcium aluminate bond. The fire protection material 27 has, for example, a weight proportion between 95% and 99% and a calcium oxide proportion between 1% and 5%. The ceramic fire protection material 27 is provided between two cover plates 29 of expandable graphite.

The sensor arrangement 1 has no fire protection housing; the ceramic fire protection material 27 adheres stably to the sensor element 3 and a cover plate 29 is glued on in upward and downward direction, respectively. The cover plates have, for example, a thickness between 1 and 3 mm and the cylindrical fire protection ceramic has a height between 40 and 60 mm. The connecting cable 8 and the plug connector 9 satisfy, for example, DIN 5510 so that a use of the sensor device 1 and the filter device (not illustrated) coupled thereto can be used in particular in situations with special safety requirements.

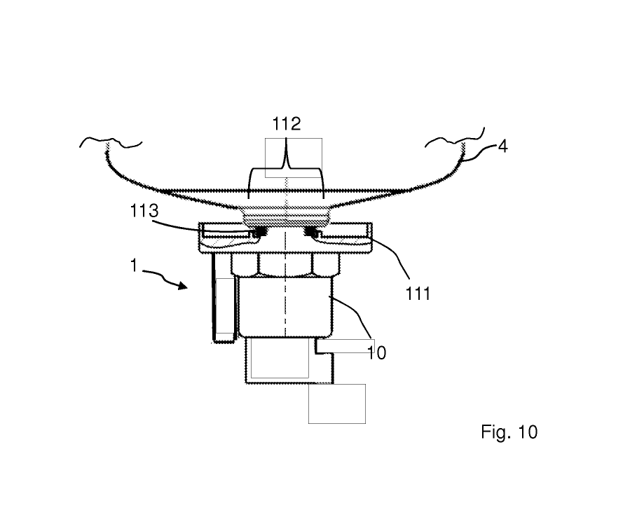

In the embodiment of a sensor arrangement 10 shown in FIG. 10, the fire protection material is a stainless steel material forming the housing 10 of the senor element and enclosing it completely. Heat that is acting on the housing 10 can be dissipated partially via the sensor element to the operating liquid contained in the interior of the filter 4 and the sensor; this delays the heating process in case of fire. In this context, the housing 10 is preferably provided with an annular disk-shaped ring projection 111 in an area adjoining the filter and projects distinctly in radial direction (i.e., at least 5 mm and preferably at least 10 mm) past the connecting area 112 of the sensor element with connecting thread (not shown) and seal 113 for sealing relative to the spin-on filter 4. In this way, flames that reach spatially from the bottom side the sensor arrangement can be deflected so that the risk of a direct contact of the connecting area 112 with the flames is significantly reduced.

In contrast to conventional sensors which are manufactured of plastic material, by providing an encasement of a fire protection layer or a fire protection material a further option of use is provided. In particular in applications in which special fire protection requirements exist, for example, in marine applications on ships, the proposed pre-line or prefilter can be used for fuels. The filter assembly and in particular the sensor arrangement fulfills in this context preferably the requirements in regard to fire resistance according to DIN EN ISO 7840 and/or ISO 19 921. In particular, fluid-tightness at the transition between the sensor element 2 and the spin-on filter 4 is achieved for at least 30 minutes at a flame temperature of 700-900° C.

Even though the present invention has been explained in more detail with the aid of preferred embodiments, it is not limited thereto but can be modified in various ways. Instantly, “one” or “a” does not preclude a plurality.

What is claimed is:

1. A sensor device for a filter assembly, the filter device comprising:
at least one sensor element configured to detect an operating parameter of the filter assembly;
a fire protection material at least partially enclosing the at least one sensor element.

2. The sensor device according to claim 1, wherein the at least one sensor element is a water sensor.

3. The sensor device according to claim 1, wherein the fire protection material comprises an intumescent material.

4. The sensor device according to claim 1, wherein the fire protection material comprises a flame-resistant fiber material.

5. The sensor device according to claim 1, further comprising a fire protection housing, surrounding the at least one sensor element, wherein the fire protection material is received in the fire protection housing so as to surround the at least one sensor element.

6. The sensor device according to claim 1, wherein a cavity is provided between the fire protection material and the at least one sensor element.

7. The sensor device according to claim 1, wherein the at least one sensor element comprises at least one connecting line configured to transmit sensor signals, wherein the at least one connecting line passes at least partially through the fire protection material to an exterior of the sensor device.

8. The sensor device according to claim 1, wherein the fire protection material is configured such that the at least one sensor element resists flame exposure of the sensor device for at least 30 minutes at at least 800° C.

9. A filter assembly comprising a filter for filtering a fluid and a sensor device according to claim 1, wherein the at least one sensor element of the sensor device is attached fluid-tightly to the filter.

10. The filter assembly according to claim 9, further comprising a seal that fluid-tightly seals the at least one sensor element relative to the filter.

11. The filter assembly according to claim 9, further comprising a securing device configured to secure the fire protection housing and/or an expanding fire protection material to the filter.

12. A method for manufacturing a sensor device according to claim 1, comprising encasing a sensor element at least partially in a fluid fire protection material that cures to a cured solidified state and is intumescent in the cured solidified state.

13. The method according to claim 12, further comprising:
positioning the sensor element in a casting mold;
metering the fluid fire protection material into the casting mold so as to enclose the sensor element by the metered-in fire protection material and/or by a foaming action of the fire protection material;
subsequently curing the fire protection material to form-fittingly encase the sensor element.

14. The method according to claim 13, wherein the fire protection material is a polyurethane material.

15. The method according to claim 13, wherein the fire protection material is connected torque-proof to the sensor element.
